(19) United States
(12) Patent Application Publication
(10) Pub. No.: US 2011/0029022 A1
(43) Pub. Date: Feb. 3, 2011

(54) SPINAL COLUMN IMPLANT

(76) Inventors: Thomas ZEHNDER, Bach (CH); Reto Braunschweiler, Neftenbach (CH)

Correspondence Address: JACOBSON HOLMAN PLLC, 400 SEVENTH STREET N.W., SUITE 600, WASHINGTON, DC, 20004, US

(21) Appl. No.: 12/844,225

(22) Filed: Jul. 27, 2010

(30) Foreign Application Priority Data
Jul. 28, 2009 (EP) 09166597.6

(51) Int. Cl. A61B 17/70

(52) U.S. Cl. 606/264

(57) ABSTRACT

A spinal column implant (1) comprises pedicle screws (2) that each are provided with a head part (5) and a screw-in part (4) that can be screwed into the respective vertebral body. Said pedicle screws (2) are connected by means of a rod-shaped connecting element (3) that includes at least partial regions that are made of an elastic material. The elastic regions can be connected in a form-fitting manner to the head parts (5) of the pedicle screws (2) and/or can be connected in a form-fitting manner to the intermediate pieces. The rod-shaped connecting element (3) is provided with a longitudinal through-hole (7), into which an essentially tensile force- and/or compressive force-resistant longitudinal element (8) is inserted, which is provided in both of its end regions with stop elements (9), (10) which can abut on the frontal end surfaces of the rod-shaped connecting element (3). Said spinal column implant allows a spinal column according to the invention to be optimally supported and stabilized.

The present invention relates to a spinal column implant comprising pedicle screws that each are provided with a head part and a screw-in part and can be screwed into the vertebral bodies by means of the screw-in parts, at least one rod-shaped connecting element that includes at least partial regions that are made of an elastic material, which, if applicable, are connected to intermediate pieces made of rigid material, whereby the pedicle screws can be connected to each other by means of said rod-shaped connecting element, whereby elastic regions can be connected in a form-fitting manner, at least partly, to the head parts of the pedicle screws and/or can be connected in a form-fitting manner, at least partly, to the intermediate pieces.

Spinal column implant of this type are inserted into spinal columns of a patient in order to stabilize said spinal column. For this purpose, the pedicle screws each are screwed into a vertebral body of the spinal column to be stabilized, the rod-shaped connecting element is inserted into the head part of the pedicle screws, the head part is sealed with a sealing facility, the rod-shaped connecting element is thus held in the respective pedicle screw. The use of a rod-shaped connecting element that includes at least partial regions that are made of an elastic material enables the vertebral bodies of the thus stabilized spinal column that are connected by means of elastic intermediate pieces to not become fully fixed, such as would be the case were a rod made of steel used, but rather a slight motion of the individual stabilized vertebral bodies with respect to each other is feasible which prevents the vertebral bodies from becoming connected to each other by ossification.

A spinal column implant of this type is known, for example, from EP1364622 B1. Said spinal column implant is used to optimally stabilize a disease-afflicted spinal column, the elastic rod is also held in the respective pedicle screws in optimal manner, which is achieved by the form-fitting connection between rod and pedicle screw. Said rod advantageously consists of a biocompatible plastic material based on polyurethane which allows optimal strength properties to be achieved at reasonable geometric dimensions.

Elastic rods of this type are not optimally suited for patients with spondylolistheses since they cannot optimally receive the forces that occur when the vertebral bodies glide forward. Neither are said rods optimally suited in cases of so-called hypermobility of a spinal column. The inserted elastic rod might, for example, be over-strained.

It is therefore the object of the present invention to provide a spinal column implant which, on the one hand, can optimally stabilize a vertebral column, but, on the other hand, also can receive the forces that may occur, for example, in cases of spondylolisthesis or hypermobility as mentioned above.

This object is met according to the invention in that the rod-shaped connecting element is provided with a longitudinal through-hole, into which an essentially tensile force- and/or compressive force-resistant longitudinal element is inserted, which is provided in both of its end regions with stop elements which can abut on the frontal end surfaces of the rod-shaped connecting element.

A spinal column implant designed as described can be used not only to provide optimal stabilizing support to the spinal column with the corresponding cushioning properties on tensile and compressive forces, but also the translational forces that may occur in particular in spondylolistheses can be received optimally.

Advantageously, at least the connecting sites by means of which the elastic regions of the rod-shaped connecting element can be connected to the head parts of the pedicle screws and/or the intermediate pieces are provided with structured surfaces which act in concert with correspondingly structured surfaces of the parts to be connected, which results in an optimal form-fitting connection.

Advantageously, the structured surfaces consist of ribs and grooves engaging each other that are easy to produce and mount.

Advantageously, the ribs and grooves are provided to be helical-shaped which allows corresponding connecting elements to be joined optimally.

Advantageously, at least one of the two stop elements of the longitudinal element is provided to be adjustable such that the tensile elongation of the rod-shaped connecting element can be limited to a maximal value.

Advantageously, the adjustable stop element is provided with an internal thread that can be screwed onto the end region of the longitudinal element, which is provided with a matching thread, and that can be fastened, whereby the option of continuous adjustment is attained.

Advantageously, the adjustable stop element is provided with a cap which overlaps the end region of the rod-shaped connecting element whereby optimal seating is attained.

Another advantageous development of the invention consists of the caps of the stop element being connected essentially in a form-fitting manner to the respective end regions of the rod-shaped connecting element that consist of elastic material. What this achieves is that not only tensile forces, but also compressive forces, can be transferred to the longitudinal element, whereby both the cushioning properties as well as the fatigue resistance properties (cold flow) of the rod-shaped connecting element can be optimized.

Advantageously, at least one of the two caps between two adjustment elements is held such as to be shiftable on the longitudinal element which results in optimal adjustment of the permissible motion range.

Advantageously, sleeves surrounding the respective elastic region of the rod-shaped connecting element are placed on the elastic regions of the rod-shaped connecting element, when these are to be inserted into the head parts of the pedicle screws, which improves the bracketing and mounting of said elastic regions in the head parts of the pedicle screws.

In order to render placing the sleeves on the rod-shaped connecting element simpler, said sleeves are formed from two halves that are connected to each other on the one longitudinal side in a hinged manner, whereas the opposite longitudinal sides serve as stop surfaces.

Advantageously, the internal surfaces of the sleeves have a structured surface that matches the structured surface of the rod-shaped connecting element, which again results in an essentially form-fitting connection being obtained.

Embodiments of the invention are illustrated in detail in exemplary manner in the following based on the appended drawings.

In the figures:

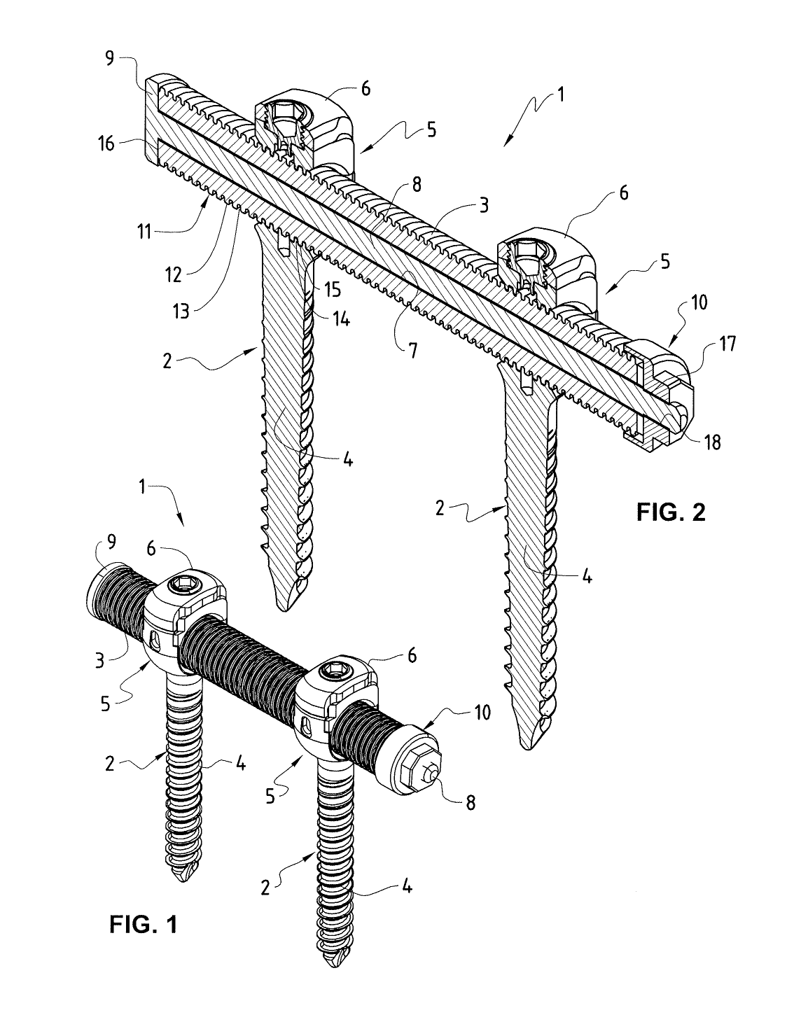

FIG. 1 shows a spatial view of a first embodiment of a spinal column implant according to the invention;

FIG. 2 shows a sectional view of the spinal column implant according to FIG. 1;

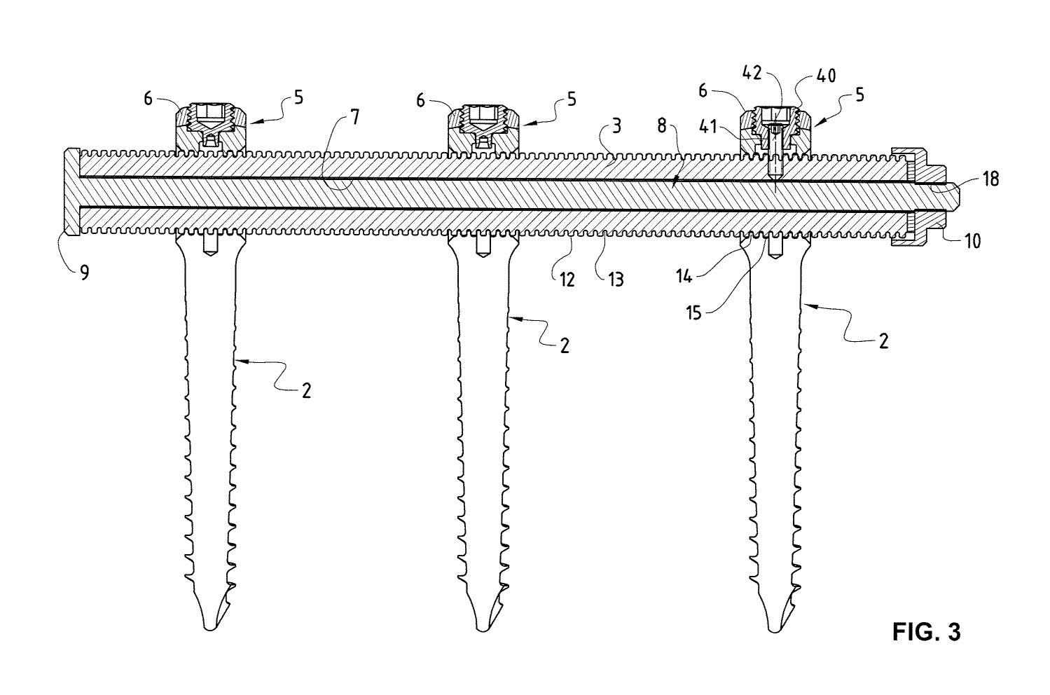

FIG. 3 shows a schematic sectional view of the spinal column implant with three pedicle screws;

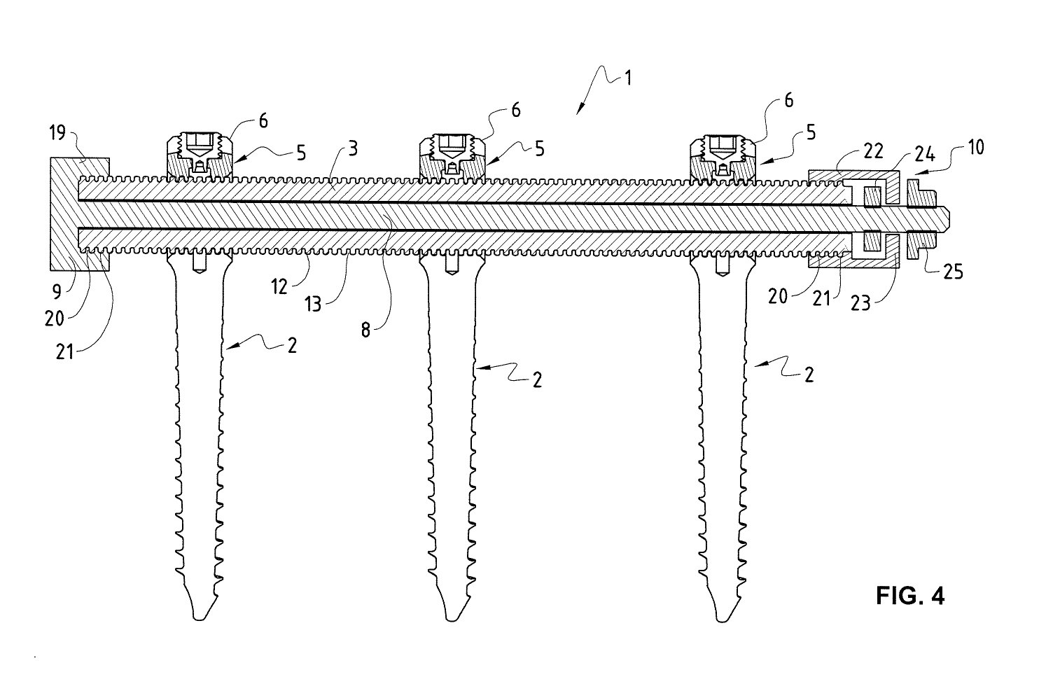

FIG. 4 shows a sectional view of a spinal column implant, in which the longitudinal element is provided with stop elements that are connected to the rod-shaped connecting element in a form-fitting manner;

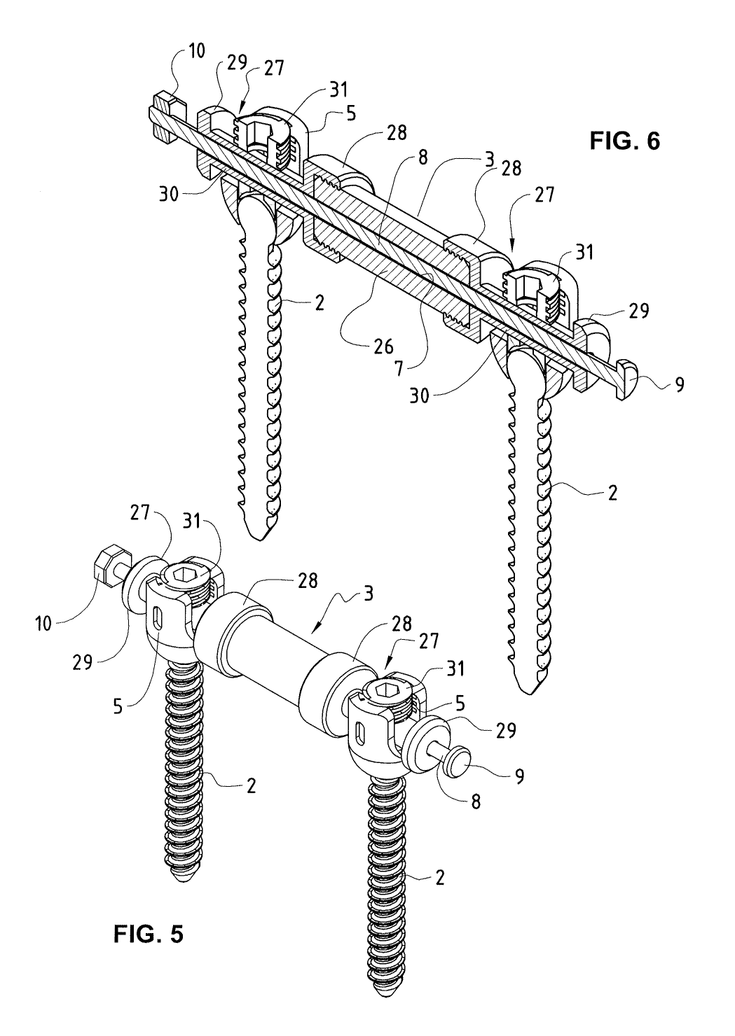

FIG. 5 shows a spatial view of a spinal column implant, in which the rod-shaped connecting element includes an elastic partial region and rigid intermediate pieces;

FIG. 6 shows a sectional view through the spinal column implant according to FIG. 5;

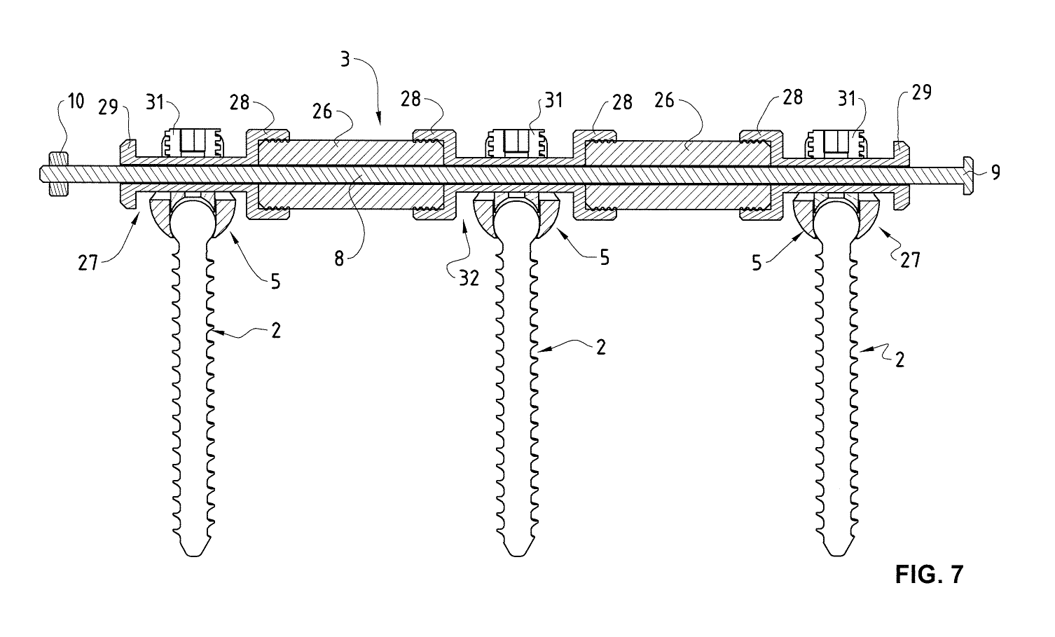

FIG. 7 shows a sectional view through a spinal column implant for three pedicle screws;

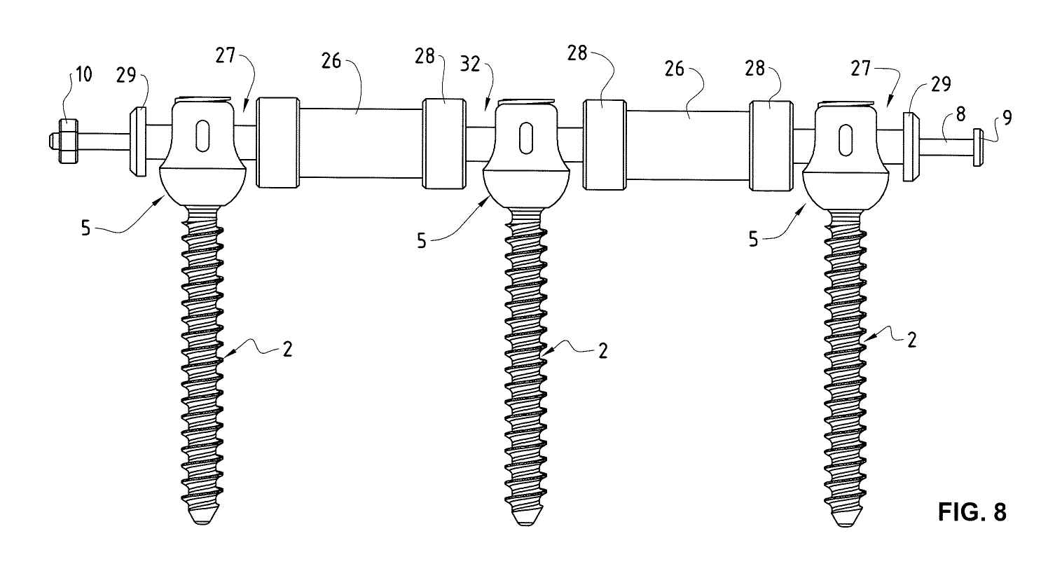

FIG. 8 shows a view onto the spinal column implant according to FIG. 7;

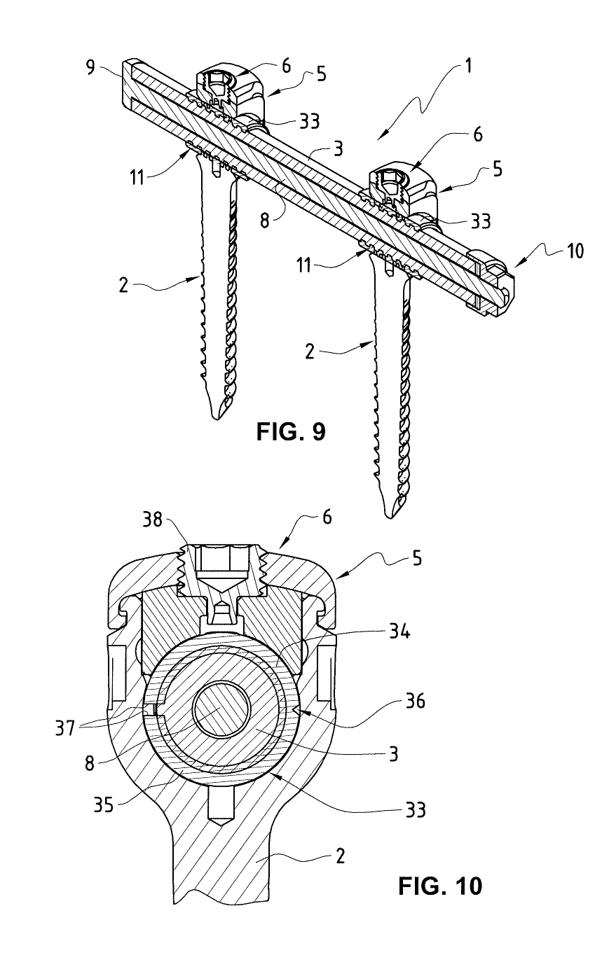

FIG. 9 shows a sectional view through a spinal column implant, in which sleeves are placed on the elastic partial regions in the region of the pedicle screws;

FIG. 10 shows a cross-sectional view of the rod-shaped connecting element that has a sleeve placed on it and is inserted into the head part of a pedicle screw;

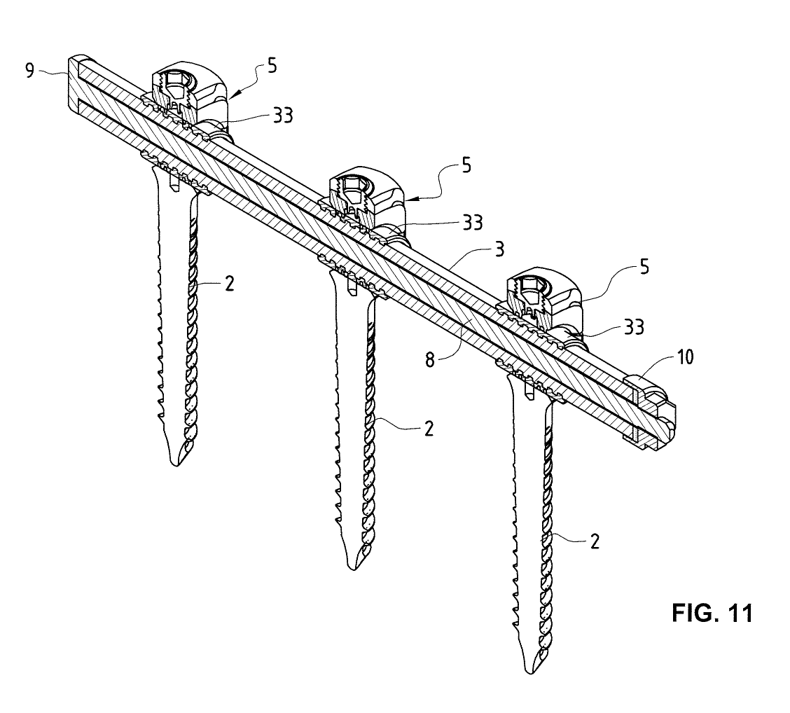

FIG. 11 shows a sectional view of a spinal column implant with three pedicle screws that has sleeves placed on the rod-shaped connecting element.

FIG. 1 shows a spinal column implant 1 that comprises two pedicle screws 2 and one rod-shaped connecting element 3. The pedicle screws 2 are each screwed into the corresponding vertebral body by their screw-in part 4, the rod-shaped connecting element 3 is inserted into the corresponding head part 5 of the pedicle screw 2 and held in said pedicle screw 2 by means of a sealing facility 6. A sealing facility of this type is described, for example, in the European patent application having the publication number, EP 2074957 A1. The rod-shaped connecting element that is inserted into the pedicle screws 2 consists of an elastic material, for example of a biocompatible plastic material based on polyurethane. A spinal column, in which a spinal column implant of this type is inserted, is supported in known manner, a certain degree of mobility of the two vertebral bodies with respect to each other is permitted.

A longitudinal element 8 is inserted into a longitudinal through-hole 7 (FIG. 2) that is provided in central position in the rod-shaped connecting element, the two end regions of longitudinal element 8 are provided with stop elements 9 and 10.

It is evident from the sectional view through the spinal column implant 1 according to FIG. 1 shown in FIG. 2 how the rod-shaped connecting element 3 is inserted into the head parts 5 of the pedicle screws 2 and is held by the sealing facilities 6. The rod-shaped connecting element 3 is provided with a structured surface 11 that is formed by ribs 12 and grooves 13 in the exemplary embodiment shown. Said ribs 12 and grooves 13 engage corresponding ribs 14 and grooves 15 that are provided in the corresponding receiving regions on the head part 5 of the pedicle screws 2 and the sealing facilities 6. Thus, a form-fitting connection is obtained in each case between the pedicle screws 2 and the rod-shaped connecting element 3 which allows the axial forces to be transferred optimally.

The longitudinal element 8 is inserted into the longitudinal bore hole 7 which extends fully through the rod-shaped connecting element 3 in axial direction. Said longitudinal element 8 is provided with a stop element 9 or 10 each at its two end regions, respectively. In the exemplary embodiment shown, the one stop element 9 is firmly connected to the longitudinal element 8, it abuts on the corresponding frontal end surface 16 of the rod-shaped connecting element 3.

The opposite stop element 10 is provided to be adjustable, it is provided with an internal thread 17 that can be used to screw the stop element onto an end region of the longitudinal element 8 that is provided with a corresponding thread 18. Said stop element 10 that is screwed onto the longitudinal element 8 can be secured in the adjusted position in known manner, for example through the use of an adhesive or in other known manner.

In its state, in which it is inserted into a spinal column, said spinal column implant 1 has the effect that the two head parts 5 of the pedicle screws 2 are moved towards each other when the human upper body is bent backward. This means that the region of the rod-shaped connecting element 3 that is situated between these two pedicle screws 2 is compressed and an accordingly cushioned motion of the spinal column is permitted. When the spinal column is bent forward, the two head parts of the pedicle screws 2 move away from each other, the region of the rod-shaped connecting element 3 that is situated between the two pedicle screws 2 is extended, the respective regions of the rod-shaped connecting element 3 that project beyond the pedicle screws 2 are compressed, since they are held by stop elements 9 and 10 of the longitudinal element 8, the resistance put up against a bending motion of the spinal column increases over-proportionally as soon as the two end regions of the rod-shaped connecting element 3 that project beyond the pedicle screws 2 touch against the stop elements 9 and 10 and are compressed. This prevents the rod-shaped connecting element 3 between the two pedicle screws 2 from being exposed to excessive tensile forces and, in addition, it prevents an excessively large motion from being permitted, whereby the end positions of said motion are cushioned in optimal manner.

The stop element 10 being adjustable allows the permitted motion of the spinal column to be limited, whereby the end positions of the motion are cushioned optimally, and this can be influenced also by being able to select the length dimensions of the regions of the rod-shaped connecting element 3 that project beyond the pedicle screws 2 and, correspondingly, the longitudinal element 8.

The rod-shaped connecting element 3 and the longitudinal element 8 with the stop elements 9 and 10 can be put together from corresponding individual components and pre-assembled, which allows the ready-made pre-assembled rod-shaped connecting element with the inserted and positioned longitudinal element to be inserted into the pedicle screws, which are screwed into the vertebral bodies, and fixed.

FIG. 3 shows a spinal column implant 1 made up according to the same structural principle as the one that was described in FIGS. 1 and 2. In the present spinal column implant 1, three pedicle screws 2 are connected to the one rod-shaped connecting element 3, a longitudinal element 8 is again inserted into the longitudinal bore hole 7 of the rod-shaped connecting element 3 and is provided on both sides with one stop element 9 and 10 each, whereby the stop element 10 again is arranged on the longitudinal element 8 such as to be adjustable. The rod-shaped connecting element 3 is again provided with ribs 12 and grooves 13 that engage corresponding ribs 14 and grooves 15 of the head part 5 of the pedicle screws 2 and a form-fitting connection is thus produced again.

Bending the upper body, and therefore the spinal column into which said spinal column implant 1 is inserted, backward, the regions of the rod-shaped connecting element 3 that are situated between the pedicle screws 2 are compressed, whereby the vertebral bodies into which the pedicle screws 2 are screwed are supported. Bending the spinal column, into which said spinal column implant 1 is inserted, forward, the regions of the rod-shaped connecting element 3 that are situated between the pedicle screws 2 are extended, the regions of the rod-shaped connecting element that project beyond the two outer pedicle screws 2 are compressed by the longitudinal element 8 and the stop elements 9 and 10 attached thereto, whereby the end position reached while bending the spinal column is cushioned again like with the spinal column implant according to FIG. 1 and FIG. 2. It is self-evident that a corresponding mode of action is also attained also if a larger number of screws is present.

FIG. 3 shows, in addition, a development for the design of the head part 5 of a pedicle screw 2. In the pedicle screw 2 shown in the top part of FIG. 3, a threaded hole 41, arranged axially, is provided in the straining screw 40 of the head part 5. A headless screw 42, which can extend through the rod-shaped connecting element 3 and can fix the longitudinal element 8 with respect to the pedicle screw 2, can be screwed into said threaded hole 41. What this achieves is that said pedicle screw 2 can serve as the base for the permissible range of motion of the longitudinal element 8. It is also conceivable that provision of said headless screws 42 in the head part 5 of the pedicle screws 2 does not result in a spinal column implant, which was inserted for mutual stabilization of the individual vertebral bodies, producing the desired outcome such that the vertebral bodies need to be fixed. With the present option, the spinal column implant that is inserted into the spinal column does not need to be replaced, but rather subsequent fixation can be attained by means of a small surgical intervention by inserting the headless screws 42.

The spinal column implant 1 as shown in FIG. 4 has the same structure as the spinal column implant 1 according to FIG. 3, with the exception of the stop elements 9 and 10 of the longitudinal element 8 being provided differently. The stop element 9 that is fixedly connected to the longitudinal element 8 includes a cap 19 that overlaps the end region of the rod-shaped connecting element 3. The overlapping region of cap 19 also includes ribs 20 and grooves 21 that engage ribs 12 and grooves 13 of the rod-shaped connecting element 3 and thus a form-fitting connection is obtained between longitudinal element 8 and rod-shaped connecting element 3. The adjustable stop element 10 also comprises a cap 22, which, in turn, overlaps the end region of the rod-shaped connecting element 3 and also is connected thereto in a form-fitting manner by means of ribs 20 and grooves 21. Said stop element 10 is adjustable again, the cap 22 comprises a fin 23 that is situated between two screw thread-featuring elements 24 and 25, which can be screwed onto the thread 18 of the longitudinal element 8, and can be secured by it in known manner. Said two screw thread-featuring elements 24 and 25, which serve as adjustment elements, can be used to adjust the shiftable range of the stop element 10.

The mode of action of said spinal column implant shown in FIG. 4 is as follows:

Bending the spinal column forward, the regions of the rod-shaped connecting element 3 that are situated between the pedicle screws 2 are extended, the two end regions of the rod-shaped connecting element 3 that project on both sides beyond the pedicle screws 2 are compressed in accordance with the embodiment according to FIG. 3, the fin 23 of the cap 22 abuts on the screw thread-featuring element 25. Bending the spinal column backward, the regions of the rod-shaped connecting element 3 that are situated between the pedicle screws 2 are compressed, in addition, the end regions of the rod-shaped connecting element 3 that project beyond the pedicle screws 2 are extended, whereby the fin 23 of the cap 22 abuts on the screw thread-featuring element 24. This achieves, in addition, cushioning of the end position for the extension motion of a spinal column.

FIGS. 5 and 6 show another spinal column implant 1, in which the rod-shaped connecting element 3 consists of an intermediate part 26 that is made of elastic material and is connected on both sides to a dumbbell-shaped element 27 that is made of rigid material. Said dumbbell-shaped elements 27 each are provided with a cap 28 that are connected to the elastic intermediate part 26 in a form-fitting manner. Each of the ends of the dumbbell-shaped elements 27 facing away from the cap 28 terminates in a flange 29. A cylinder-shaped region 30 is provided between cap 28 and flange 29, said cylinder-shaped region 30 is inserted into the head part 5 of the pedicle screws 2 in each case and clamped tight in the pedicle screw in known manner by means of clamping screws 31. The rod-shaped connecting element 3 with the two dumbbell-shaped elements 27 again is provided with a longitudinal through-hole, into which the longitudinal element 8 is inserted, which again is provided with a stop element 9 that is firmly connected to the longitudinal element 8, and an adjustable stop element 10.

The functional principle of the spinal column implant 1 shown in FIGS. 5 and 6 corresponds to what is shown in FIGS. 1 and 2: bending the spinal column forward, the elastic intermediate part 26 of the rod-shaped connecting element 3 is extended, whereby the end position is defined by the stop elements 9 and 10 of the longitudinal element 8; bending the spinal column backward, the elastic intermediate part 26 is compressed, whereby the cushioning support for the spinal column is attained again. Since the cross-section of the cylinder-shaped region of the rod-shaped connecting element 3 in the region of the pedicle screw head can be smaller than the cross-section of the elastic intermediate piece 26, known polyaxial pedicle screws can be used as well.

FIG. 7 shows a spinal column implant that corresponds to the spinal column implant 1 as shown in FIGS. 5 and 6, whereby said spinal column implant comprises three pedicle screws 2. A dumbbell-shaped element 27 each is inserted into the two outer pedicle screws 2 and clamped tight by means of the clamping screws 31, the middle pedicle screw 2 has a dumbbell-shaped element 32 inserted into it that includes a cap 28 each on both sides by means of which said dumbbell-shaped element 32 is connected on both sides to the elastic intermediate parts 26 in a form-fitting manner.

The functional principle of said spinal column implant 1 that is shown in FIG. 7 essentially corresponds to what is shown in FIG. 3: bending the spinal column backward, the elastic intermediate parts 26 are compressed, whereby a cushioning support for the spinal column is attained, and bending the spinal column forward, the elastic intermediate parts 26 are extended, whereby the maximal bending motion of the vertebral bodies, into which the pedicle screws 2 are inserted, is limited by the stop elements 9 and 10 of the longitudinal element 8.

FIG. 8 shows a view onto the spinal column implant 1 according to FIG. 7 with equivalent elements being identified by the same reference numbers.

FIG. 9 shows a spinal column implant 1 that corresponds to the spinal column implant that is shown in FIGS. 1 and 2 with the sole exception being that the rod-shaped connecting element 3 that consists of elastic material is provided with a structured surface not over its entire length, but said structured surface 11 being restricted to the regions that are subject to the influence of the pedicle screws 2. In this context, a sleeve 33 each is placed on the rod-shaped connecting element 3 in the region of the pedicle screws 2, by means of the structured surfaces of the sleeves 33 and of the rod-shaped connecting element 3, an essentially form-fitting connection results in this case as well. This solution is known from European patent application having application number 08156089.8. A clamping connection is obtained between sleeve 33 and head part 5 of the pedicle screw. The use of said sleeves 33 simplifies the assembly of the rod-shaped connecting element 3, the surface of the rod-shaped connecting element in the region of the sleeve can be either smooth or structured in this context. In this context, the structuring of the surface can be limited to the region of the sleeve or it can extend over the entire length of the rod-shaped connecting element. The effect of the use of a sleeve 33 is that the pressure peaks of the clamping forces in the plastic material can be minimized. Moreover, the longitudinal bore hole in the rod-shaped connecting element is not deformed excessively, the desired functional principle is ensured. A solely compressive connection is attained between the head part of the pedicle screw and the sleeve, which leads to very easy assembly. As before, standard pedicle screw can be used for a sleeve of normal diameters.

The sectional view according to FIG. 10 shows the head part 5 of the pedicle screw 2, into which the rod-shaped connecting element 3 with an inserted longitudinal element 8 is inserted. The sleeve 33 is placed around the rod-shaped connecting element 3, whereby the sleeve 33 consists of two halves 34 and 35 that are connected to each other on one longitudinal side via a hinge 36 such that they can be swiveled. On the opposite longitudinal side, the respective halves 34 and 35 of the sleeve 33 each include a stop surface 37 each, which abut on each other in the fully tensioned state of the clamping screw 38 of the sealing facility 6. This ensures that the elastic rod-shaped connecting element 3 is not compressed too strongly in the fully clamped position.

FIG. 11 shows a spinal column implant 1 that corresponds to the one shown in FIG. 3 whereby a sleeve 33 each, as described in detail in conjunction with FIG. 10, is inserted in the connection region of pedicle screw 2 and rod-shaped connecting element 3. The functional principle of said spinal column implant corresponds to that of the spinal column implant that has been shown in and described in conjunction with FIG. 2.

All of the spinal column implants described above comprise a rod-shaped connecting element that is provided with a longitudinal through-hole 7 into which the longitudinal element 8 is inserted. All rod-shaped connecting elements 3 can slide on the respective longitudinal element 8. A suitable material can be selected in each case for the longitudinal element 8 depending on the strain to which said longitudinal element 8 will be subjected. The material can, for example, be a titanium alloy, in particular if said longitudinal element 8 is subjected to both tensile and compressive forces and if corresponding translational forces have to be received. However, said longitudinal element 8 can also consist of a suitable plastic material, in particular if only small translational forces have to be received, whereby said material should possess at least relatively high tensile strength. Moreover, said longitudinal element can also be made of a cable or rope made of a metallic or plastic material (e.g. polyester), in particular if said longitudinal element is only to be subjected to tensile forces. Said spinal column implants according to the invention allow this feature to be adapted according to need in each case, whereby optimal support for and stabilization of the corresponding spinal column can be attained.

1. A spinal column implant comprising pedicle screws that each are provided with a head part and a screw-in part and can be screwed into the vertebral bodies by means of the screw-in parts, at least one rod-shaped connecting element that includes at least one partial region that is made of an elastic material, which, if applicable, are connected to intermediate pieces made of rigid material, whereby the pedicle screws can be connected to each other by means of said rod-shaped connecting element, whereby the elastic region can be connected in a form-fitting manner, at least partly, to the head parts of the pedicle screws and/or can be connected in a form-fitting manner, at least partly, to the intermediate pieces, characterized in that the rod-shaped connecting element is provided with a longitudinal through-hole, into which an essentially tensile force- and/or compressive force-resistant longitudinal element is inserted, which is provided in both of its end regions with stop elements which can abut on the frontal end surfaces of the rod-shaped connecting element.

2. The spinal column implant according to claim 1, characterized in that at least the connecting sites by means of which the elastic regions of the rod-shaped connecting element can be connected to the head parts of the pedicle screws and/or the intermediate pieces are provided with structured surfaces which act in concert with correspondingly structured surfaces of the parts to be connected and provide a form-fitting connection.

3. The spinal column implant according to claim 2, characterized in that the structured surfaces consist of ribs and grooves which engage each other.

4. The spinal column implant according to claim 3, characterized in that the ribs and grooves are provided to be helical-shaped.

5. The spinal column implant according to claim 1, characterized in that at least one of the two stop elements of the longitudinal element is provided to be adjustable.

6. The spinal column implant according to claim 5, characterized in that the adjustable stop element is provided with an internal thread that can be screwed onto the end region of the longitudinal element, which is provided with a matching thread, and can be fastened.

7. The spinal column implant according to claim 5, characterized in that the adjustable stop element is provided with a cap which overlaps the end region of the rod-shaped connecting element.

8. The spinal column implant according to claim 7, characterized in that the caps of the stop elements are connected essentially in a form-fitting manner to the respective end regions of the rod-shaped connecting element that consist of elastic material.

9. The spinal column implant according to claim 8, characterized in that at least one of the two caps between two adjustment elements is held such as to be shiftable on the longitudinal element.

10. The spinal column implant according to claim 1, characterized in that sleeves surrounding the respective elastic region of the rod-shaped connecting element are placed on the elastic regions of the rod-shaped connecting element, when these are to be inserted into the head parts of the pedicle screws.

11. The spinal column implant according to claim 10, characterized in that the sleeves are formed from two halves that are connected to each other on the one longitudinal side in a hinged manner, whereas the opposite longitudinal sides serve as stop surfaces.

12. The spinal column implant according to claim 10, characterized in that the internal surfaces of the sleeves essentially have a structured surface that matches the structured surface of the rod-shaped connecting element.
